(19) United States
(12) Patent Application Publication
(10) Pub. No.: US 2008/0294228 A1
(43) Pub. Date: Nov. 27, 2008

(54) METHOD AND DEVICE FOR CONTROLLED STIMULATION OF LYMPHATIC FLOW

(75) Inventors: M. Jason Brooke, Minneapolis, MN (US); Allan C. Shuros, St. Paul, MN (US); Robert J. Sweeney, Woodbury, MN (US); Alok S. Sathaye, Boston, MA (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, St. Paul, MN (US)

(21) Appl. No.: 11/752,377

(22) Filed: May 23, 2007

(51) Int. Cl. A61N 1/05

(52) U.S. Cl. 607/116

(57) ABSTRACT

A device and method for controllably augmenting the flow of lymphatic fluid through one or more lymphatic vessels. The device may utilize various means of modulating the flow of lymph, including neural, mechanical and/or chemical stimulation and could be a stand-alone device or be incorporated into any cardiac, neuromodulation and/or drug delivery device.

### RELATED APPLICATIONS

This application is related to co-pending U.S. patent application Ser. Nos. 11/422,414, filed on Jun. 6, 2006; Ser. No. 11/422,417, filed on Jun. 6, 2006; Ser. No. 11/422,418, filed on Jun. 6, 2006; Ser. No. 11/422,423, filed on Jun. 6, 2006; Ser. No. 11/422,421, filed on Jun. 6, 2006; and Ser. No. 11/539,301, filed on Oct. 6, 2006, hereby incorporated by reference.

### FIELD OF THE INVENTION

This invention pertains to methods and systems for treating disease with implantable devices.

### BACKGROUND

The lymphatic system and the cardiovascular system are closely related structures that are joined by a capillary system. Lymph, or lymphatic fluid, is the fluid that seeps outside the blood vessels in interstitial spaces of body tissues and is then absorbed by lymphatic capillaries to flow back into the bloodstream through the lymphatic vessels. The terminal structures of the lymphatic vessels include the right lymphatic duct, which drains lymph fluid from the upper right quarter of the body above the diaphragm and down the midline, and the thoracic duct, located in the mediastinum of the pleural cavity which drains the rest of the body. The lymphatic system performs two primary functions: 1) it drains excess fluids and protein from interstitial tissues to maintain a proper distribution of fluids and nutrients in the body, and 2) it facilitates the action of the body's immune system by filtering out organisms that cause disease and producing lymphocytes that attack foreign organisms and generate antibodies. Through the flow of blood in and out of arteries, into the veins, and through the lymph vessels and nodes, the body is thus able to eliminate the products of cellular breakdown and bacterial invasion.

As aforesaid, one of the functions performed by the lymphatic system is the conveying back to the blood of fluid and proteins exuded from the blood vessels into the interstitial space. Exuded fluid and proteins are absorbed by lymphatic capillaries and then flow into the venous system through the lymphatic vessels. The lymphatic system is normally very efficient at removing excess fluid from the interstitial space and is even able to maintain a slight negative pressure. Under certain conditions, however, the lymphatic system is so overwhelmed with fluid that a buildup occurs, referred to as edema. Edema may be due to dysfunction of the lymphatic system in transporting fluid such as may occur when the lymphatic vessels become obstructed (e.g., elephantiasis) or may be due to excess fluid in the interstitial space such as may occur in heart failure (e.g., pulmonary edema), kidney disease, liver disease, cancer, and infectious disease.

It is believed to be beneficial for lymphatic flow to be increased in patients having edema due to any of the causes listed above. In addition, the rate of lymphatic flow has been shown to be positively correlated with improving health in patients with many other diseases (e.g., pneumonia). Presumably, this is because the lymphatic vessels also communicate with lymph nodes and facilitate the body's immune function by transporting foreign antigens to the lymph nodes from the interstitial spaces. Increasing lymphatic flow via manual manipulation techniques has long been practiced by osteopathic physicians and has been shown to reduce illness time and hospitalization in patients with a number of diseases. Certain types of physical exercise may also increase lymphatic flow through muscular compression of the lymphatic vessels. Manual manipulation techniques and physical exercise are limited, however, in their effectiveness in increasing lymphatic flow.

### BRIEF DESCRIPTION OF THE DRAWINGS

FIG. 1 illustrates the physical placement of an implanted lymphatic stimulation device.

FIGS. 2A and 2B illustrates the components of exemplary lymphatic stimulation devices.

FIG. 3 is a system diagram of an exemplary lymphatic stimulation device.

FIGS. 4A through 4C illustrate different embodiments of a lymphatic flow stimulator.

### DETAILED DESCRIPTION

This disclosure relates to a device and method for controllably augmenting the flow of lymphatic fluid through one or more lymphatic vessels. The device may utilize various means of modulating the flow of lymph, including neural, mechanical and/or chemical stimulation and could be a stand-alone device or be incorporated into any cardiac, neuromodulation and/or drug delivery device. Such a device may be useful in patients having pathological lymph flow activity from any cause, including heart failure, pulmonary edema, cancer, elephantiasis, and/or other disorders.

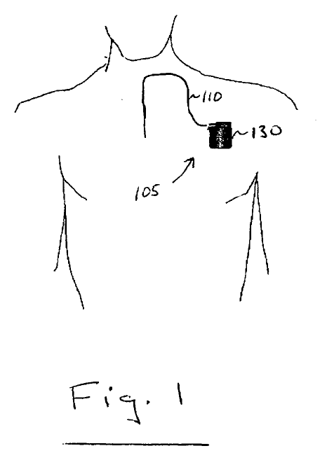

FIG. 1 shows an exemplary physical placement of an implantable device for controlled lymphatic stimulation as described herein. In one embodiment, an implantable device 105 is placed subcutaneously on the patient's chest or abdomen, similar to a standard cardiac pacemaker. The device is connected to a lead 110, a distal portion of which incorporates a sensor for sensing a physiological parameter related to lymphatic flow, referred to herein as a lymphatic sensor. The lead and lymphatic sensor are implanted within the lymphatic system using a venous approach that involves initial entry into the venous blood system. In the embodiment depicted in FIG. 1, the lead 110 passes subcutaneously from the device housing 130 to a point of venous access in the upper chest or neck such as the subclavian vein. The lead is then guided into the thoracic duct ostium using standard fluoroscopy techniques in order to position the lymphatic sensor at a selected location within the lymphatic system. Techniques for introducing leads, catheters, and similar instrumentation into the lymphatic system are described in detail in the aforementioned U.S. patent application Ser. No. 11/422,423, filed on Jun. 6, 2006, incorporated by reference. An alternative implantation approach includes placing the lymphatic sensor using a direct surgical approach. A flow stimulating means may also be incorporated into the lead 110 or may be incorporated into a separate lead or catheter that is connected to the device 105 and similarly implanted into the lymphatic system at a selected location.

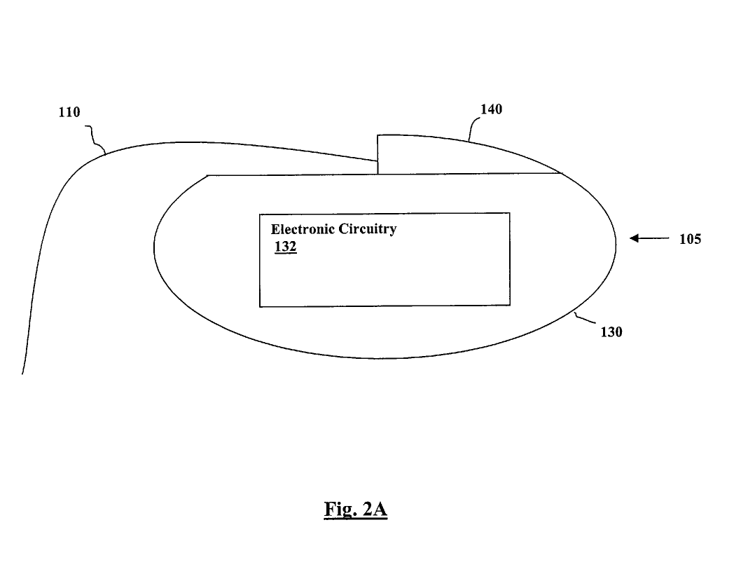

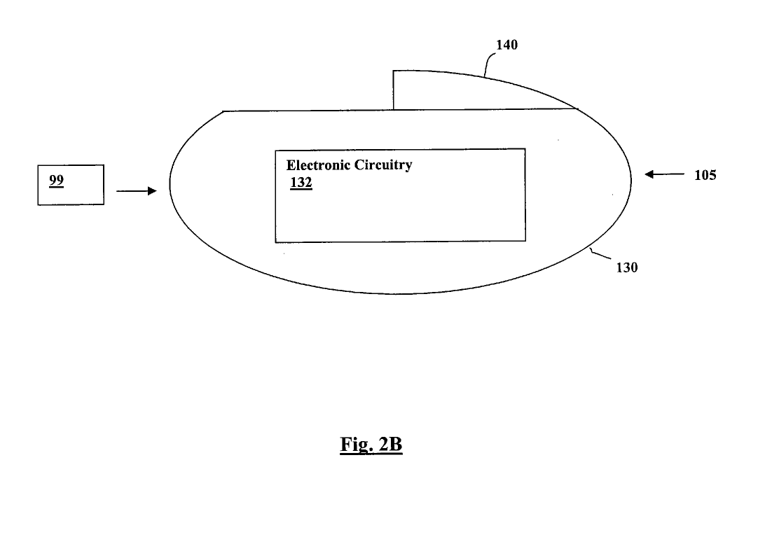

FIG. 2A shows the basic components of the device 105 in more detail. The device 105 includes a hermetically sealed housing 130 that is placed subcutaneously or submuscularly in a patient's chest or other convenient location as noted above. The housing 130 may be formed from a conductive metal, such as titanium, and may serve as an electrode for delivering electrical stimulation. A header 140, which may be formed of an insulating material, is mounted on the housing 130 for receiving one or more leads and/or a catheter 110 that are adapted for implantation into a lymphatic vessel. The lymphatic sensor may be incorporated into a lead 110 that is electrically connected to the electronic circuitry 132 within the housing that includes circuitry for monitoring lymphatic flow. The device also incorporates a lymphatic flow stimulator actuated by the electronic circuitry 132 such as: 1) one or more electrodes incorporated into a lead 110 or a different lead and connected to pulse generation circuitry within the housing, 2) a mechanical stimulator incorporated into a lead or a catheter 110 and connected to an actuator within the housing, and/or 3) a catheter 110 connected to a pump or other drug delivery apparatus within the housing. In alternative embodiments, the lymphatic sensor and/or lymphatic flow stimulator may be a separately implantable unit that communicates with the electronic circuitry 132 via wireless telemetry. FIG. 2B illustrates an exemplary device in which the lymphatic sensor is incorporated into a stent 99 adapted for implantation in a lymphatic vessel or a blood vessel. The stent 99 and the electronic circuitry 132 in this embodiment include telemetry circuitry for wireless communication.

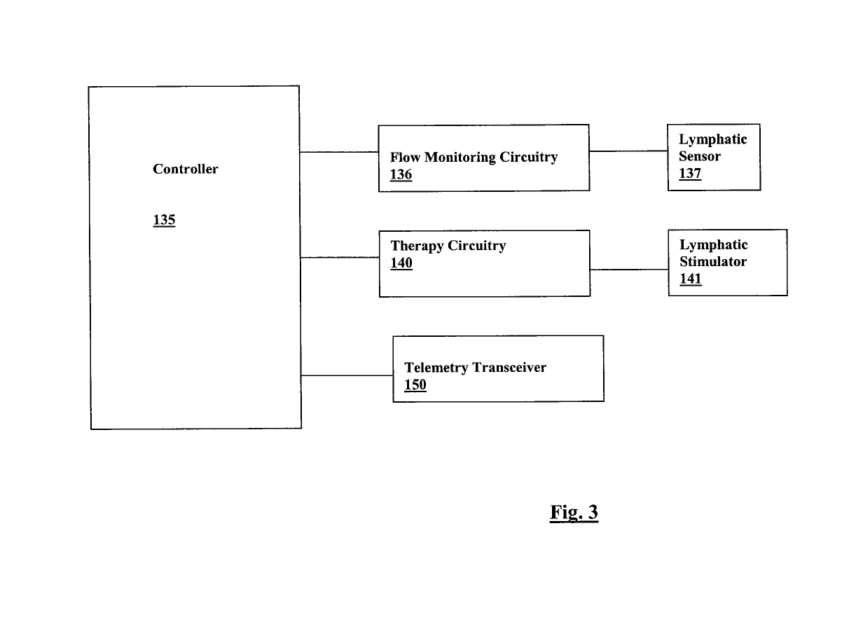

Contained within the housing 130 are the components for providing the functionality to the device as described herein which may include a power supply (not shown), flow monitoring circuitry 136, therapy circuitry 140, and a programmable electronic controller 135 for controlling the operation of the device. FIG. 3 is a system diagram of the functional components of the device. The controller 135 may be made up of discrete circuit elements but is preferably a processing element such as a microprocessor together with associated memory for program and data storage which may be programmed to perform algorithms for delivering therapy. (As the terms are used herein, “circuitry” and “controller” may refer either to a programmed processor or to dedicated hardware components configured to perform a particular task.) The controller is interfaced to flow monitoring circuitry 136 from which it receives data generated by one or more lymphatic sensors 137. The monitoring circuitry may include, for example, circuitry for amplification, filtering, and/or analog-to-digital conversion of voltages generated by a lymphatic sensor. The controller 135 is also interfaced to therapy circuitry 140 in order to control the action of lymphatic flow stimulator 141 in response to conditions sensed by the flow monitoring circuitry. The controller may then be programmed to actuate the flow stimulator to control lymphatic flow in a closed-loop fashion. For example, the flow stimulator may be actuated in response to a decrease in lymphatic flow below a specified limit value as measured by the lymphatic sensor. Flow stimulation may then be applied for a specified period of time or continuously. In the latter case, the flow stimulation may be ceased when the lymphatic flow increases above the same or another specified limit value. Flow stimulation could also be applied according to a defined schedule, where the duty cycle of the flow stimulation modified in accordance with cumulative or average flow measurements taken over some specified period of time.

Also interfaced to the controller in FIG. 3 is a telemetry transceiver 150 capable of communicating with an external programmer or a remote monitoring device. An external programmer enables a clinician to receive data and modify the programming of the controller, and a remote monitoring device may be further interfaced to a network (e.g., an internet connection or telephonic network) for communicating with a patient management server to allow clinical personnel at remote locations to receive data from the remote monitoring device as well as issue commands. The controller may be programmed such that, when particular conditions are detected by the monitoring circuitry (such as when a measured parameter exceeds or falls below a specified limit value), the device transmits an alarm message to the remote monitoring device and to the patient management server to alert clinical personnel.

The lymphatic sensor 137 is a sensor for sensing a parameter related to fluid flow within a lymphatic vessel and generating a flow signal therefrom. In various embodiments (which embodiments may be used alone or in combination) the lymphatic sensor may be a sensor for measuring current induction due to ionic flow in electric fields, a Doppler flow sensor, a pressure sensor for measuring lymph vessel pressure changes (intra- and/or trans-vessel), a mechanical sensor for directly measuring fluid flow within a lymphatic vessel, or a chemosensor for measuring changes in the molecular concentration of particular ions or bio-marker that are related to fluid flow. Depending upon the embodiment, the lymphatic sensor may be incorporated into a lead that is implanted into a lymphatic vessel, intravenously implanted near a lymphatic vessel, or otherwise surgically implanted. The lymphatic sensor could also be a sensor measuring a parameter indirectly related to lymphatic flow such as neural or muscle activity measurements.

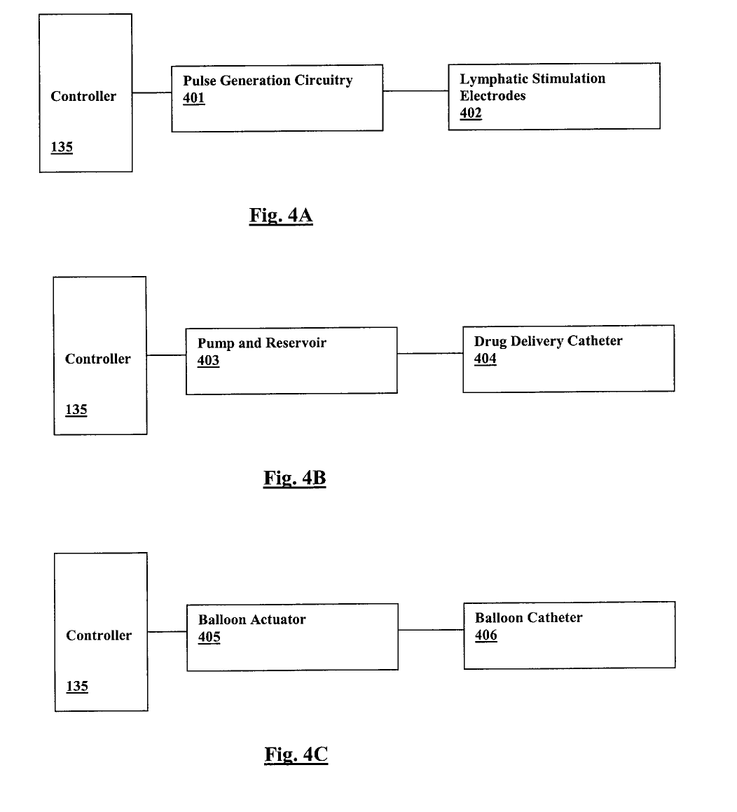

The lymphatic flow stimulator 141, as controlled by the controller and therapy circuitry, acts to augment the flow of lymphatic fluid within a lymphatic vessel. The lymphatic stimulator may be implemented according to various embodiments, which embodiments may be used alone or in combination in a particular device. In one embodiment, illustrated in FIG. 4A the lymphatic flow stimulator is incorporated into a lead and comprises one or more electrodes 402 connected to pulse generation circuitry 401 interfaced to the controller 135 within the housing for delivering electrical pulses to the smooth muscle of the lymphatic vessel and cause contraction thereof. The frequency and/or amplitude of the pulses are controlled by the controller and may be modulated in accordance with lymphatic fluid flow measurements. Either instead of, or in addition to, stimulation of lymphatic vessel smooth muscle, the lymphatic flow stimulation electrodes may stimulate nerves that innervate lymphatic vessels to cause contraction thereof. The lymphatic flow stimulation electrodes may be disposed either within or outside of a lymphatic vessel.

In another embodiment, the lymphatic flow stimulator 141 comprises a drug delivery device actuated by the controller. As illustrated in FIG. 4B, such a drug delivery device may comprise a controller-actuated pump and associated drug reservoir(s) 403 located within the housing 130 that communicate with the lumen of a drug delivery catheter 404. The drug delivery catheter 404 may be implanted within a lymphatic vessel with its distal opening for discharging the chemical agent at a selected location. The chemical agent is preferably selected to be one or more short-acting, reversible agents that cause contraction and/or relaxation of the lymphatic vessel smooth muscle. Examples of such agents include stimulation/depolarization agents such as acetylcholine or other cholinomimetic agents (i.e. cholinergic agonists or cholinesterase antagonists), serotonin, histamine, dopamine, noradrenaline, nitric oxide and certain proteins. When actuated to stimulate lymphatic flow, the drug delivery device may deliver a vessel dilating agent to promote collection of fluid within the vessel, a vessel constricting agent to cause contraction of the vessel and movement of fluid therethrough, or may deliver both types of agents. In the latter case, the drug delivery device may deliver a vessel dilating agent followed by a vessel constricting agent to enhance the pumping action of the lymphatic vessel.

In another embodiment, the lymphatic flow stimulator 141 is a mechanical stimulator of the lymph vessel incorporated into an implantable catheter or lead, where the mechanical stimulator may be located in the lymphatic vessel or adjacent to the vessel. Mechanical stimulation may be applied in the form of fluid pressure or vibrations to the lymphatic vessel smooth muscle in order to cause contraction thereof. Fluid pressure may be created in the vessel by inflating a balloon at the distal end of a catheter or by injecting fluid into the lymphatic vessel out of the distal end of the catheter. In an exemplary embodiment illustrated in FIG. 4C, a balloon actuator 405 is interfaced to the controller 135 for controllably inflating a balloon catheter 406. In another embodiment, the mechanical stimulator is piezo-electric crystal that vibrates the tissue to cause contraction. Other types of acoustic or ultrasonic transducers could be also be used.

An exemplary embodiment of a device for controllably stimulating lymphatic flow as described above would include an implantable housing, monitoring circuitry contained within an implantable housing, a lymphatic sensor interfaced to the monitoring circuitry for sensing a parameter related to fluid flow within a lymphatic vessel and generating a flow signal therefrom, a controller interfaced to the monitoring circuitry for processing flow signals generated by the lymphatic sensor, and a lymphatic flow stimulator interfaced to the controller. The controller is then programmed to actuate the flow lymphatic flow stimulator in response to changes in the flow signal. For example, the controller may be programmed to actuate the lymphatic flow stimulator if the flow signal decreases below a specified value. The device may also be equipped with a telemetry transceiver interfaced to the controller for enabling wireless communication with the controller, and the controller may be programmed to transmit an alarm message via the telemetry transceiver if the flow signal generated by the lymphatic sensor exhibits a specified change. The lymphatic sensor may be incorporated in a lead connected to the monitoring circuitry, where the lead is adapted for implantation into or near a lymphatic vessel. Alternatively, the lymphatic sensor may be adapted for implantation into or near a lymphatic vessel and be equipped with a telemetry transceiver for communicating with the monitoring circuitry via wireless telemetry. In the latter case, the lymphatic sensor may incorporated into a stent adapted for implantation within a lymphatic vessel or a blood vessel. The lymphatic sensor may be a sensor of a type selected from a group that includes a pressure sensor, an ultrasonic flow sensor, a mechanical flow sensor, inductive flow sensor, and a chemo-sensor for generating a voltage proportional to the concentration of a particular chemical species in the lymphatic fluid. In one embodiment, the lymphatic flow stimulator comprises a lead having one or more stimulation electrodes incorporated therein adapted for disposition near smooth muscle of the lymphatic vessel or nerves innervating the smooth muscle. In another embodiment, the device includes a drug reservoir and a pumping mechanism interfaced to the controller within the implantable housing, and the lymphatic flow stimulator comprises a drug delivery catheter connected to the pumping mechanism, the drug delivery catheter adapted for disposition near smooth muscle of the lymphatic vessel or nerves innervating the smooth muscle to deliver a vessel dilating and/or constricting agent thereto. In another embodiment, the lymphatic flow stimulator is a mechanical stimulator for causing contraction of lymphatic vessel smooth muscle. For example, the lymphatic flow stimulator may be a balloon incorporated into a catheter connected to a balloon actuator within the implantable housing.

The invention has been described in conjunction with the foregoing specific embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. A device, comprising:
an implantable housing;
monitoring circuitry contained within an implantable housing;
a lymphatic sensor interfaced to the monitoring circuitry for sensing a parameter related to fluid flow within a lymphatic vessel and generating a flow signal therefrom;
a controller interfaced to the monitoring circuitry for processing flow signals generated by the lymphatic sensor; and,
a lymphatic flow stimulator interfaced to the controller, wherein the controller is programmed to actuate the flow lymphatic flow stimulator in response to changes in the flow signal.

2. The device of claim 1 wherein the lymphatic sensor is incorporated in a lead connected to the monitoring circuitry, the lead being adapted for implantation into or near a lymphatic vessel.

3. The device of claim 1 further comprising a telemetry transceiver and wherein the lymphatic sensor is adapted for implantation into or near a lymphatic vessel and communicates with the monitoring circuitry via wireless telemetry.

4. The device of claim 3 wherein the lymphatic sensor is incorporated into a stent adapted for implantation within a lymphatic vessel or a blood vessel.

5. The device of claim 1 wherein the lymphatic sensor is a sensor of a type selected from a group that includes pressure sensor, an ultrasonic flow sensor, a mechanical flow sensor, inductive flow sensor, and a chemo-sensor for generating a voltage proportional to the concentration of a particular chemical species in the lymphatic fluid.

6. The device of claim 1 further comprising a telemetry transceiver interfaced to the controller for enabling wireless communication with the controller and wherein the controller is programmed to transmit an alarm message via the telemetry transceiver if the flow signal generated by the lymphatic sensor exhibits a specified change.

7. The device of claim 1 wherein the lymphatic flow stimulator comprises a lead having one or more stimulation electrodes incorporated therein adapted for disposition near smooth muscle of the lymphatic vessel or nerves innervating the smooth muscle.

8. The device of claim 1 further comprising:
a drug reservoir and a pumping mechanism interfaced to the controller within the implantable housing; and,
wherein the lymphatic flow stimulator comprises a drug delivery catheter connected to the pumping mechanism, the drug delivery catheter adapted for disposition near smooth muscle of the lymphatic vessel or nerves innervating the smooth muscle to deliver one or more chemical agents thereto, wherein the one or more chemical agents are selected from a group that includes vessel dilating agents and vessel constricting agents.

9. The device of claim 1 wherein the lymphatic flow stimulator is a mechanical stimulator for causing contraction of lymphatic vessel smooth muscle.

10. The device of claim 9 wherein the lymphatic flow stimulator is a balloon catheter connected to a balloon actuator within the implantable housing.

11. A method, comprising:
implanting a housing having monitoring circuitry and a controller contained therewithin;
implanting a lymphatic sensor interfaced to the monitoring circuitry for sensing a parameter related to fluid flow within a lymphatic vessel and generating a flow signal therefrom;
implanting a lymphatic flow stimulator interfaced to the controller,
programming the controller to actuate the flow lymphatic flow stimulator in response to changes in the flow signal.

12. The method of claim 11 wherein the lymphatic sensor is incorporated in a lead connected to the monitoring circuitry, the lead being implanted into or near a lymphatic vessel.

13. The method of claim 11 wherein the lymphatic sensor is implanted into or near a lymphatic vessel and communicates with the monitoring circuitry via wireless telemetry.

14. The method of claim 13 wherein the lymphatic sensor is incorporated into a stent and implanted within a lymphatic vessel or a blood vessel.

15. The method of claim 11 wherein the lymphatic sensor is a sensor of a type selected from a group that includes pressure sensor, an ultrasonic flow sensor, a mechanical flow sensor, inductive flow sensor, and a chemo-sensor for generating a voltage proportional to the concentration of a particular chemical species in the lymphatic fluid.

16. The method of claim 11 further comprising programming the controller to transmit an alarm message via a telemetry transceiver if the flow signal generated by the lymphatic sensor exhibits a specified change.

17. The method of claim 11 wherein the lymphatic flow stimulator comprises a lead having one or more stimulation electrodes incorporated therein and further comprising disposing the lymphatic flow stimulator near smooth muscle of the lymphatic vessel or nerves innervating the smooth muscle.

18. The method of claim 11 wherein the lymphatic flow stimulator comprises a drug delivery catheter connected to a pumping mechanism and further comprising disposing the drug delivery catheter near smooth muscle of the lymphatic vessel or nerves innervating the smooth muscle to deliver one or more chemical agents thereto, wherein the one or more chemical agents are selected from a group that includes vessel dilating agents and vessel constricting agents.

19. The method of claim 11 wherein the lymphatic flow stimulator is a mechanical stimulator for causing contraction of lymphatic vessel smooth muscle.

20. The method of claim 19 wherein the lymphatic flow stimulator is a balloon catheter connected to a balloon actuator within the implantable housing.
